(19) United States
(12) Patent Application Publication
(10) Pub. No.: US 2011/0262536 A1
(43) Pub. Date: Oct. 27, 2011

(54) METHOD TO AUTHENTICATE GENUINE TABLETS MANUFACTURED BY COMPRESSING POWDER

(75) Inventors: Frederic Jordan, Les Paccots (CH); Martin Kutter, Remaufens (CH); Celine Di Venuto, Bossonnens (CH)

(73) Assignee: ALPVISION S.A., VEVEY (CH)

(21) Appl. No.: 13/141,933

(22) PCT Filed: Dec. 22, 2009
(86) PCT No.: PCT/EP2009/067724

§ 371 Date: Jun. 23, 2011

(30) Foreign Application Priority Data
Dec. 23, 2008 (EP) 08172867.7

(51) Int. Cl. A61K 9/20; A61P 43/00; A61K 9/00; G06K 9/00; B29C 59/02

(52) U.S. Cl. 424/464; 382/141; 264/119; 424/400

(57) ABSTRACT

A method to authenticate genuine tablets manufactured by compressing powder between a punch/die set comprising the steps of: creating a microstructure on the surface of at least one of the face of the punch/die set; compressing the powder between the punch and the die; acquiring at least one reference image of the face of the punch/die set containing the microstructure or of a face of a tablet corresponding to the microstructure; acquiring at least one test image of a tablet to be authenticated; computing a level of similarity by an electronic device between the at least one test image and the at least one reference image; comparing the computed level with a threshold value so as to define if the acquired tablet is genuine.

### INTRODUCTION

The present invention concerns the field of tacking and authenticating genuine products such as tablets or pills manufactured by compressing powder.

### PRIOR ART

Most of the tablets used in the pharmaceutical industry are manufactured by compressing powder between a so-called punch and a die. The recognition of the medication is mainly based on the package, once the tablet is removed from the blister, it is very difficult to known exactly which medication is contained in the tablet. A first solution is to shape the punch or the die so that a recognizable visual element helps the user to recognize the name of the medication. Since the surface is small, the visual element is limited to generally one character.

Another solution is to apply a reference on the tablet by an edible ink. This solution is used on tablet having a coating.

It has been noticed that the counterfeited tablets or pills contain also such recognizable pattern. Those patterns are no deterrent for the counterfeiters and in fact serve only the medical people dealing of a lot of different tablets so that they do not mix two medications.

### SHORT DESCRIPTION OF THE INVENTION

The purpose of this invention is to provide a method to recognize tablets or pills by authenticate elements, those elements being very difficult to reproduce for the counterfeiters.

Accordingly, the present invention proposes a method to authenticate genuine tablets manufactured by compressing powder between a punch/die set comprising the steps of:

at an initial stage:
creating a microstructure on the surface of at least one of the face of the punch/die set,
compressing the powder between the punch and the die,
acquiring at least one reference image of the face of the tablet for which the punch/die set contains microstructure,

And at a later stage:
acquiring at least one test image of a tablet to be authenticated which is supposed to contain microstructure,
computing a level of similarity by an electronic device between the test image and each of the reference image,
comparing the computed level with a threshold value so as to define if the acquired tablet is genuine.

This invention describes methods for obtaining tablets/pills having a surface featuring microstructures that can be automatically recognized by software processing of the digital image of the surface. A given microstructure is obtained on the tablet surface by modifying the punch tool. Therefore, the invention focuses on two particular sets of methods: methods for designing the punch tool and methods for automatically recognizing the fingerprint image. Although the rest of the invention focuses on the punch, exactly the same concepts described hereafter also apply to the die, or in a combination in which the modifications are applied to the punch and to the die. The reference image will be taken on the surface of the tablet for which the tool contains microstructure. In case that the punch and the die contains microstructure, two reference images will be stored in relation of the manufacturing process obtains by this tool.

### SHORT DESCRIPTION OF THE FIGURES

The present invention will be better understood thanks to the attached figures in which:

FIG. 1 shows how is computed the roughness of surface.

FIG. 2 shows the Fourier spectrum corresponding to a white noise signal.

FIG. 3 shows the effect on the Fourier spectrum of tablets alteration caused by manufacturing and handling processes.

FIG. 4 shows how the spectrum of the punch can be designed in order to compensate for the tablet alterations.

FIG. 5 illustrates how a specific design of the tablet can protect the fingerprint area against chocks between tablets.

FIG. 6 describes the methodology used to optimize the parameters of the punch manufacturing process.

FIG. 7 describes the methodology used to optimize the parameters of the punch manufacturing process, taking into account the alterations related to the finishing of the tablet.

FIG. 8 shows how the robustness of the detectability can be evaluated by measuring the width of the cross-correlation peaks, as a function of the rotation angle.

FIG. 9 shows 3 different methods for acquiring a digital image of the surface of the tablet (A) with a digital scanner, (B) with a microscope and (C) with a hand-held microscope.

FIG. 10 describes a system enabling to acquire a digital picture of a tablet using specular reflection and to automatically position the tablet using a vibrating device.

FIG. 11 shows how the reflected light can be correlated with the orientation of a specular microstructure. In (A) the reflected light is medium and the surface is perpendicular, in (B) the surface is tilted to left and the reflected light is maximized and in (C) the surface is tilted to the right and there is not reflected light.

FIG. 12 describes how a circular surface can be warped onto a rectangle defined in horizontal by the sampling angle and in vertical by the sampling radius. An alternate representation uses the logarithm of the radius in order to obtain invariance in respect of scaling.

FIG. 13 shows a punch and a close-up of the surface of the part used to compress the powder which features a random microstructure.

FIG. 14 shows a close-up of a tablet compressed with this tool and the microstructure of the punch that has been transferred on it.

FIG. 15 shows the whole process for creating tablets and registering reference images.

FIG. 16: Diagram describing the detection strategy progressively increasing cross-correlation sizes. The first Set S0 contains X0 candidates of size 2n. The candidates that have an SNR which is superior to t1 are classified in X12. Those which have an SNR which is inferior to t1 are classified in X22. The set S1 contains the X12 candidates of size 2n+1. The same matching is performed at each step. The last set Sx should contain only one candidate.

FIG. 17 shows the way Fourier coefficients (complex values) can be stored in the database. The coefficients displayed in black are stored in each column 491 of the database table 493. The figure shows that column 1 has only one coefficient (the average value of the image), the column 2 has the 3 following coefficients, the column 3 has 12 coefficients, etc. . . . This approach enables to optimize the required bandwidth for transferring data (492) from the database on the hard disk to the CPU. A new line 494 is allocated in the database for each reference image.

FIG. 18 shows the coverage of the database size using the “Best Rank” method. For each set of images of a given size, a certain number Cixp of items should be correlated. Cixp follows a geometrical law. During the detection process, the common ratio of this law is increased until Cix1 is bigger than Card (S0).

FIG. 19 shows an image of a counterfeit tablet on the left and an image of a genuine tablet on the right. In this picture the height of A character is smaller in the counterfeit tablet.

### DETAILED DESCRIPTION

### Manufacturing Process

### Objectives

The manufacturing process must be designed in such a way that each tablet (in this document we mainly use the word tablet/tables, however, it is a placeholder for any similar item, such as pills, etc) features a microstructure with the following properties:
Machine recognizable: The microstructure should be such that it must be possible to reliably recognize it by comparing its digital image to a set of reference images. This sets some constraints on the microstructure depth and size, such that various alterations due to manufacturing and handling of tablets do not prevent successful identification.
Challenging to counterfeit: The counterfeit operation consists in replicating the microstructure of the tablet. If the microstructure uses small variations of depth and size, than it will be more challenging to duplicate. If those modulations reach the same order of magnitude as the particles which constitute the tablet, duplication will be even more difficult, as it will be challenging to differentiate between random variations related to particle distribution and random variations caused by the punch. In case modulations are smaller than noise caused by particle distribution, then counterfeit will become extremely challenging.
Invisible: The microstructure should not alert the consumer (neither potential counterfeiter), so the structure of the microstructure should be made as uniform and natural as possible.

For the remaining part of the description, the term “reference image” refers to the image of the tablet acquired at the manufacturing stage. The term “test image” refers to the image acquired in the field, when a tablet should be authenticated.

### Pill Design

The various parameters characterizing the manufacturing and composition of the tablet have an influence over the reproduction of the random structure obtained by the punch surface.

For instance, the average grain size of the powder can be related to the highest frequency of the noise structure that can be obtained. In addition, the manufacturing process by itself may not reproduce exactly the original noise texture of the punch, depending on the sticking coefficient of the powder.

Moreover, several other stages of the manufacturing process may also degrade the detectability of the microstructure. This is for instance the case for the process of tablet coating, during which a layer is applied around the tablet. This layer may alter the image of the microstructure as it can flatten it and add some random noise on each tablet.

Therefore, depending on the thickness of the coating, the defects of the microstructure have to be larger, so that the microstructure can still be recognized through the coating. The coating process itself, during which tablets collide between them can also mechanically modify this microstructure, alter the image and add some random noise to each tablet. For this reason the reference image can be acquired before the coating process instead of after, in order to obtain a basis, which is common to all the coated tablets, damaged or not.

Finally, handling and image acquisition also introduce alterations (for instance, the tablet is not flat in most of the cases which impacts on the quality of the digital image of the tablet surface). One solution is to take into account the depth of field of the acquisition device, which has to be such that the microstructure can still be detected even if the surface of the tablet is not flat. Another solution is to use only part of the tablet as a reference and as a test image, this part being as flat as possible.

Since the core idea of the whole approach consists in leaving, as much as possible, the tablet manufacturing process unchanged, it is necessary to apply specific strategies in order to compensate the effect of those alterations on the fingerprint detectability and on the manufacturing of the punch die set. There are basically two different kinds of strategies: optimization of the punch/die design and optimization of the detection algorithm.

### Punch Design

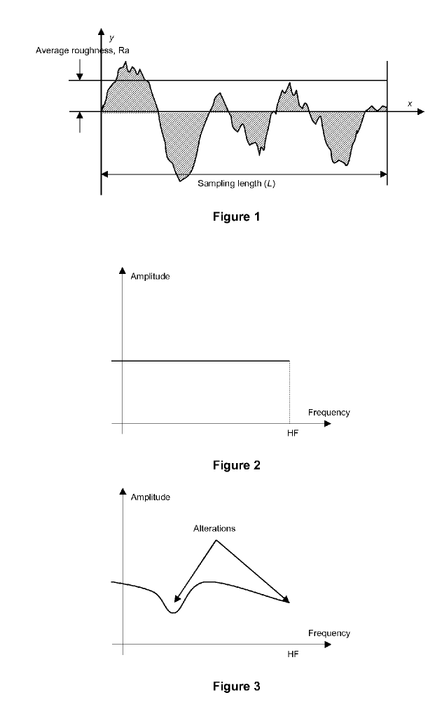

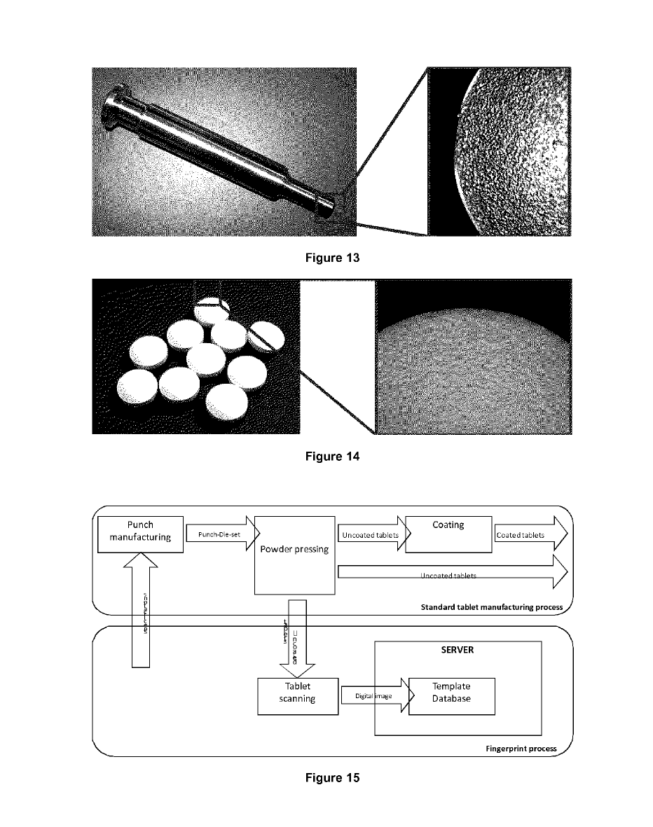

Tablets punch/die sets are typically made of metallic alloys which shape is obtained usually using machining or electro-erosion, but other techniques like molding, laser, plasma, arc, drilling, oxy-fuel, hydro abrasion, chemical etching can also be used. The goal of the design techniques described below is to obtain a punch with some specific microstructure properties. FIG. 13 shows an example of the microstructure of the surface of a punch/die. While compressing the powder, the microstructure will be transferred and reproduced on the tablet. A picture of tablets produced with the punch of FIG. 13 can be seen in FIG. 14. In the field of machining, there already exists a concept of so-called roughness, Ra, which defines the microstructure property by the equation (see FIG. 1):

$$Ra = \frac{1}{L} {\int }_{0}^{L} \vert y ( x ) \vert d x$$

It should be noted that other definition of the surface microstructure can also be described using other parameters like maximum valley depth, maximum peak height, skewness, kurtosis, etc. . . . and the current invention is not limited to one specific measurement technique.

In order to obtain such noisy surface on the punch/die, the following techniques can be used:
Electro-erosion or Electro Discharge Machining (EDM): This technique enables to simultaneously machine the shape of punch and to obtain a given roughness of the surface. Machining is obtained by removing matter using high voltage sparks which erode the surface. The machined matter is typically a metallic alloy. The sparks generate high temperatures which results in craters in the machined matter. The sizes of the craters depend on several parameters, including in particular the current intensity, the gap voltage and the electrical pulses durations. The numbers and depth of these craters define the roughness of the surface. This surface roughness (typically measured in Ra or Charmilles units) may be corrected by specific surface treatments but it may also be a desirable property of the surface (which is often the case for moulds of plastic parts). It is also particularly useful in the case of the disclosed invention, since punch manufacturers using EDM are able to control the grain of the random texture which is created on the surface of the machined punch.
Sanding or sandblasting: This technique enables to create some roughness at the surface of a mould by blasting sand (or other materials) on it.
Deposit: Another approach consists in the deposit of non-glossy additional material on the surface of the punch. For instance, a powder can be deposited and stick on the punch surface by various means, including inter-diffusion processes.
Others: Basically, any shaping technique can be used in order to obtain a noisy surface. Indeed, most of the shaping techniques create defects which can be used for fingerprinting. For instance, hydro, chemical etching or laser abrasion will create such defects.

The various properties of the tablet powder and the whole tablet manufacturing process may substantially impact the detectability of the fingerprint. For instance, a powder made of large rounded grains will typically have less high-frequency details than a powder made of small grains. The same applies for the chemical properties of the powder, the shape of the tablet, the kind of metal coatings used for the punch, the pressure applied, etc. Since punch tools must be manufactured for each type of tablet to be protected, it is useful to define a methodology enabling to quickly and efficiently define the optimal parameters used to create the punch (types of machining process, size of the grains of the fingerprint created on the punch, etc). As an example FIG. 14 shows the example of tablet microstructure created with a punch.

Typically, the microstructure of the punch should be designed such that the powder follows the microstructure. In order to obtain accurate images of the microstructure, the average size of the defects creating the microstructure is most of the time between 5 to 20 um.

FIG. 6 describes an efficient methodology: an image acquisition device is used to obtain a digital image of the microstructure area of the tablet. This area can be part of the tablet or be the whole tablet. This image is then analyzed in order to evaluate if it can be efficiently used for microstructure application. This efficiency can be evaluated by using different parameters including in particular the following ones:
Robustness to rotation: This parameter evaluates if the microstructure can still be detected when the reference image is slightly rotated versus the reference image. The FIG. 8 shows two examples of detection signals versus the rotation angle. With the correct angle, the detection signal reaches approximately 1. With other angles, the signal drops rapidly. The example on the right of FIG. 8 shows a rotation robustness which is higher compared to the example on the left. It can be mathematically characterized by the width L of the signal at 50% of the maximum of the signal, the larger the value, the higher the robustness. One possibility to increase the size of L is to increase the average size of the defects in the microstructure.
Robustness to cropping: Robustness against cropping means that detection can be successful even with a fraction of the reference image. This can be evaluated by computing the detectability obtained for different crop sizes. Assuming that the detectability is represented by a value d, then this detectability is a function d(s) which decreases when s decreases. Robustness can be defined by a value Rc according to the following equation:

$$If s > Rc then d ( s ) > \frac{Max ( d ( s ) )}{T}$$

T is a constant value. For instance T=2, means that Rc defines the cropping size below which the detectability is half of the maximum detectability. To some extent, this method can be used to compute the robustness when part of the full size image is damaged, letting only a certain percentage to perform the authentication.
Shape of the Fourier spectrum: It is also possible to characterize the detectability by computing a scalar value which represents the deviation between the amplitude of the Fourier transform of the microstructure image and a reference Fourier spectrum. This reference can for instance be the spectrum of an ideal white noise, which is equal to a constant value for all frequencies. In such a case, the value characterizing the efficiency of the microstructure can be obtained simply by computing the standard deviation of the amplitudes of the Fourier transform.
Robustness to generic alteration: It is possible to characterize the microstructure efficiency by evaluating the ability of the microstructure image to be invariant to some defined alterations. These alterations could be physical or due to the imaging device.
Examples of physical alterations are:
   A coating layer which is applied on the tablet
   The sticking coefficient of the powder that can make that sometimes the powder remains stuck in the tool instead of being transferred to the tablet.
   The coating process in which the tablets are chocked together and can be damaged up to a certain point.
Examples of alterations due to the imaging device are:
   Scale distortion
   Directional geometrical distortions
   Fish eye distortions
   Lighting aberrations

### Optimization of the Punch Design to Compensate for Alterations

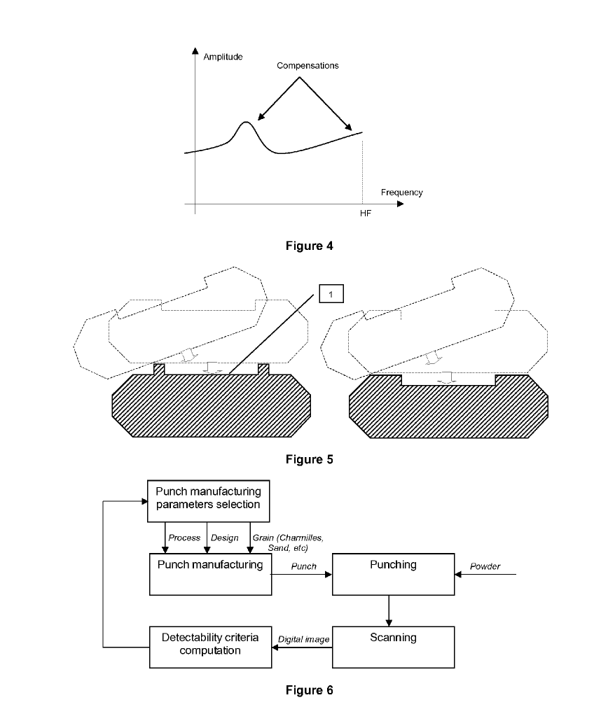

The optimization of the punch design consists in defining the best parameters for creating the noisy/grainy texture of the punch such that final tablet can be easily detected after that all the finishing process is completed. This finishing process introduces many alterations to the surface microstructure which decreases the detectability (for instance—but not limited to—powder characteristics, coating parameters, etc). One solution consists in optimizing the punch design such that those alterations will have a minor impact on the detectability. Two different approaches can be considered in the optimization of the punch: alteration compensations based on analysis in the frequency domain and alteration prevention based on particular design strategies of the punch.
Frequency analysis approach: Frequency domain analysis is performed by performing Fourier transform of the digital image of tablets made with a noisy punch. We assume in the following that the optimal detectability of a known noise is reached for white noise signals (FIG. 2). However, the described process can also apply basically to any kind of signal statistics. Each alteration is associated with spectral modifications. For instance, wearing of the punch during operations may lead to a smoother punch surface, and therefore to a decrease in the energy for the highest frequencies of the spectrum (an example on the spectrum is shown in FIG. 3). Knowing this effect, it is possible to compensate during the design of the punch in order to have more energy for higher frequencies (FIG. 4). This methodology can be basically applied for any kind of signal alteration which can be characterized in the frequency domain.
Specific design strategies: The design can also be optimized by improving the morphological design of the tablet. For instance, during the tablet coating process, many tablets are put in a processing chamber where they basically float using high speed air streams. This process leads to many chocks between the tables which may alter their surface, and therefore also the fingerprint that was left by the punching process. However, it is possible to circumvent to some extent such alterations by designing a special punch shape which leads to protect tablets against these chocks. FIG. 5 shows two examples where the microstructure surface (1) is protected against some types of collisions between tablets thanks to a specific design of the tablet shape. It can be noted that in these examples, there still exist some cases where a colliding tablet can damage the microstructure surface (1). It is possible to mathematically model the probability that two colliding tablets will damage the microstructure surface, given the 3D description of the tablet shape (and even evaluate the type of resulting defects on the fingerprint surface, depending on the shape of the tablet part which comes in physical contact with the fingerprint surface). It is also important to notice that the detectability of the microstructure depends on the shape and the area of the microstructure surface. Typically, designs efficiently protecting against alteration will have smaller fingerprint areas and thus have a decreased detectability. Therefore, the design strategy is a trade-off between detectability and alteration protection.

The above-mentioned optimization techniques rely on the following methodology:
Punch manufacture
Tablet manufacture
Detectability of the obtained microstructure
Finishing
Detectability of the final microstructure,
Comparison between both detectability
Refinement of the punch manufacturing process

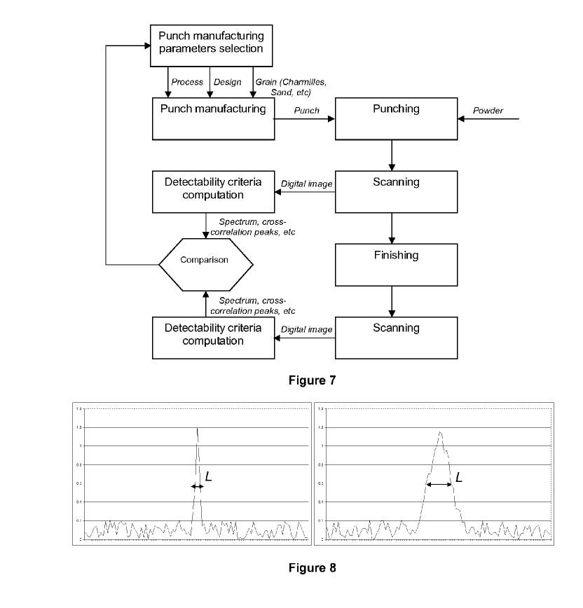

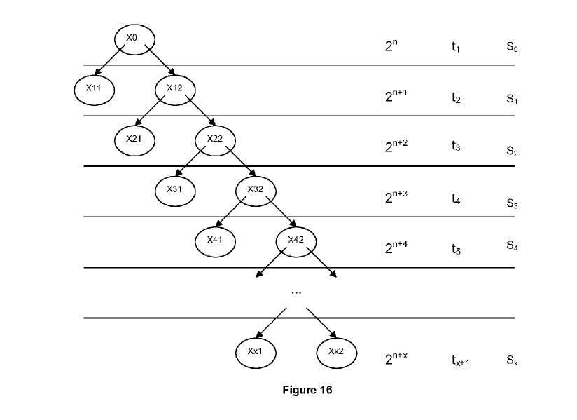

This methodology is schematically described in FIG. 7.
Punch surface design strategies: Yet another strategy in order to detect successfully the microstructure after coating consists in selecting a roughness of the punch that is sufficiently high in order to be successfully detected after coating, as shown in FIG. 15. For instance, one basic rule consists in using a punch/die roughness (or any other microstructure measurement as defined later in this document) which is proportional to thickness of the coating (for instance a roughness equal to half or twice the coating thickness). Moreover the reference image of the tablet may preferably be done before coating, since this typically leads to a higher detection signal. The powder grain size may also influence the way the punch microstructure is transferred on the tablet. One approach consists in designing the punch microstructure such that the smallest holes or peaks are at least twice larger than the grain size (using either average size or largest size for instance).

### Imaging Process

### Objective

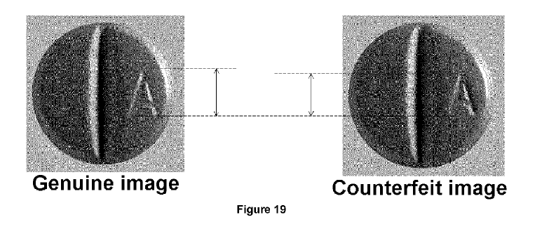

The imaging process consists in creating a digital image of the surface of the microstructure of the punch or of the tablet. These images are used for two different processes:
Creation of reference images: The surface of the punch/die or the surface of the tablet can both be used for the reference images. Using the punch/die image will theoretically lead to the best results (as the obtained image will not be disturbed by the noise of the tablet which is different for each tablets since it depends on the unique configuration of the powder particles), and should be used whenever as possible. However, there are some cases, where the reference image should be taken from the pills: highly reflective punch/die finishing leading to problematic light reflections, organizational/logistical considerations (people managing the reference images may not have access to the punch/die tools but only to the tablets) or any other effect related to punch/die or imaging that could lead to significant differences between the images of the punch/die and of the image of the tablet (like stretching or deformations for instance). The reference image can be taken from either both faces of the tablets (or from punch and die) or the reference can be taken from only one face (punch or die) of the tablet or the punch/die set. If only one side is imaged as a reference, it is possible to determine which side of the tablet to be authenticated to scan by adding a macroscopic design on one side of the tablet. A macroscopic design is a design that can by easily recognized by a naked eye. This macroscopic design can also be used to determine the rotation angle. The quality of the reference image can be validated by trying to compare it to itself in different orientations. As described in FIG. 8, the reference should match with itself rotated only if the rotation angle is smaller than a given value. In any case, it is not possible to obtain more than 1 peak in a figure like FIG. 8. In this case, the reference will be rejected. In a powder compression machine, there are generally between 40 and 60 punch die set. All the sets should be protected in order to protect all the pills created by the machine by taking a reference image of each punch/die set or a reference tablet produced by this punch/die set. The database storing the reference images will then store a set of reference images, at least one reference image per punch/die set. In addition, the macroscopic design can also be used to automatically recognize a tablet brand (this also applies to different dosages and more generally to any other subset) and restrict the authentication to the set of reference images corresponding to this brand. Finally, the macroscopic image can also be used as an authentication feature. Indeed, when tablet are counterfeit, the logos or text on the tablet are often incorrectly reproduced by the counterfeiter. It is therefore possible to compare the test image to a reference image and output a similarity level of the designs in order to authenticate a tablet, a similarity level below a given threshold being used as an indication of a counterfeit. Typical differences are height of character, width of characters, position of marks. These differences can be between them and relative to tablet borders. Other differences between genuine and fake tablets are orientation angles between mark and depth of engraving. FIG. 19 shows that the height of the characters can be different on genuine and counterfeit images.
Authentication: The authentication can only be performed by imaging the tablet surface (unless this is the punch itself that should be authenticated). As the microstructure is very precise, some “dust” on the tablet or on the imaging device can degrade the quality of the authentication process. Furthermore, if the “dust” was already present on the imaging device when the reference was acquired can lead to false positive detection. To avoid this, it is possible to digitally remove this “dust” once the image is acquired (reference or test image to be tested). This is done by replacing the value of the pixels which are too different from the mean value of the tablet image by the mean value or by random noise with the same statistics (mean value, standard deviation . . . ) than the tablet image. Another possibility to avoid false positive authentication is to use the same process as the reference validation described above: the image should match with the reference rotated only if the rotation angle is smaller than a given value. In any case, it is not possible to obtain more than 1 peak in a figure like FIG. 8. In this case, the tablet will not be considered as genuine.

### Principle of Surface Imaging

The described invention relies on the capability of an imaging device to digitally record the imperfections, defects, micro-accidents or irregularities of a tablet surface. It is therefore critical to understand how such measurement can be obtained with an imaging device. Basically, two effects are used to measure the shape of the surface, shadows and specular reflections. The FIG. 11 schematically shows a magnified view of the profile of a surface tablet. A light emitter and a light receiver are also shown for 3 different orientations cases. It can be seen that in case (A), the detector records a low level of light intensity corresponding to the so-called diffuse reflection phenomenon. In case (B) the angles are such that much more light is reflected (generally the maximum of light is reflected for this angle), it is a particular case called specular reflection. In the last case (C) the incident light does not even reach its target since it is casted by another accident on the surface, and the reflected light is therefore equal to zero. This illustrates how the micro shape of the surface can be recorded using a standard imaging system. It should be observed that the described configuration of light emitter and detector corresponds to diffuse imaging system, which basically means that in case (A) (flat surface) the reflection is of diffuse type. Other configurations exist; in particular the co-focal illumination consists in having the light emitter and the light detector at the same location. In such configurations, a specular reflection would occur in case (A) and a diffuse reflection would occur in case (B). In practice, this means that probing a perfect mirror with such a configuration would result in a white image, while doing so with a diffuse reflection configuration would lead to a black image.

In all configurations, the measured light intensity is related to the angle of the reflector and therefore the obtained image characterizes the shape of the examined surface.

Finally, although it was shown that there is a relation between the obtained digital image and the micro-topography of the sample, it is important to understand that some factors can seriously disturb this relation. For instance, if two pictures of the same tablet (or the reference tablet and the test tablet) are taken with the incident light coming from two different directions, the obtained images will be significantly different. Ideally, there should not be any differences since the micro-topology of a tablet does obviously not depend on the illumination system. This is for instance the case of digital scanners where the angle of the incident light will depend on the rotation angle of the tablet on the scanner. One solution consists in trying to infer the shape of the surface knowing the incident light angle using so-called shape from shading techniques. Such techniques take as input one or several digital images of the sample and compute the elevation map of the sample.

### Standard Acquisition Devices

One of the imaging devices combining both a large availability on the market and a good imaging performance is the document scanner. Indeed, off-the-shelf scanners typically feature 1200 dpi to 2400 dpi optical resolution which is enough to resolve details of 20 to 10 micrometers. Moreover, it is also possible to use low resolution scans in order to determine where the tablet is on the scanner before performing a high resolution scan of this area. Finally, it should be noted that scanners work by measuring the diffuse reflectivity.

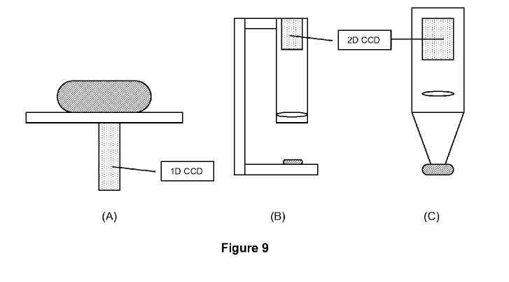

The aforementioned scanners can be characterized by the fact their principle is based on the motion of a 1D CCD (charge coupled device) over the area to be imaged (see FIG. 9-A). On the contrary, there many devices, also readily available, which include imaging system based on 2D CCD and do not require moving parts, particularly interesting examples of such devices for the described applications are:

Microscopes: Optical microscopes can be equipped with a 2D CCD in order to obtain a digital image of the observed area (FIG. 9-B). Microscopes typically provide for a very high resolution of several thousand dpi. Moreover, some of them also include some special lighting or filtering devices which can increase the quality of the obtained image. In particular, co-focal lighting, polarization filter and colored lighting can typically greatly enhance the contrast of the image. Recently has appeared a new generation of small microscopes sold as “USB microscopes” which can be connected to USB port of PC. Visualization is provided by a software application running on the PC which displays the captured image on the connected monitor. Such devices have the advantage of being much more affordable. However, they have the drawback of being less convenient to use. Indeed, the device must generally be handheld, which requires precise and delicate positioning. In general there is a physical contact between the sample and the microscope where the surface is examined (FIG. 9-C) or a physical contact between the surface on which the sample is lying and the microscope. This enables to control, to some extent, the angular positioning of the device in respect of the sample.

Digital cameras: Resolution of recent digital cameras in the consumer market combined with Macro mode enable to reach effective resolution well over 600 dpi. It is therefore possible to use such devices for fingerprint applications. Since the device is hand held, and since there is typically no physical contact between the camera and the sample, the positioning (distance between camera an object) and orientation (angle between sample surface and camera) is subject to a high degree of variability between successive test images. Moreover, the lighting is less controlled compared to the lighting obtained with microscopes and documents scanners. For all these reasons, digital camera is an acquisition device that is complex to use for fingerprinting applications. However, despite these difficulties, it remains a very interesting device since many mobile phones are equipped with such cameras. This enables in particular to provide in one unique device the 3 following functionalities:

Image capture: The image can be captured using the camera of the mobile phone. In order sufficiently high resolution, a macro mode and an autofocus are typically required. Moreover, many mobile phones also include flash illumination, which is often required in order to obtain sharp images.

Image upload: The captured image can be uploaded to a dedicated server (by MMS or email attachment for instance). This server will contain the reference images of all set of punch/die set used to produce the tablet. Non only the punch/die set currently used for the production are stored but also the punch/die set that was used before and replaced by a new punch/die set. Each time a new punch/die set is installed on the production device, a new reference image (or images is both faces are taken into consideration) is stored into the database of the server. In order to limit the comparison process between the test image and the reference images, the user can input a medication name (or identifier of the medication) of the tablet he supposes to have. The comparison will then executed with the reference images for that medication only which are related to the identifier.

Detection result display: The server can send back the result of the microstructure analysis and display it (SMS or email by instance) or even play specific audio signals or ring tones (using ring-tone associated with specific number, MMS or audio email attachment for instance).

### Specific Acquisition Devices

It is possible to design specific acquisition devices in order to optimally image the surface of a pill. In particular a tailored made design enable to overcome many of the issues encountered with off-the-shelf acquisition devices:

Stability and Angular Orientation

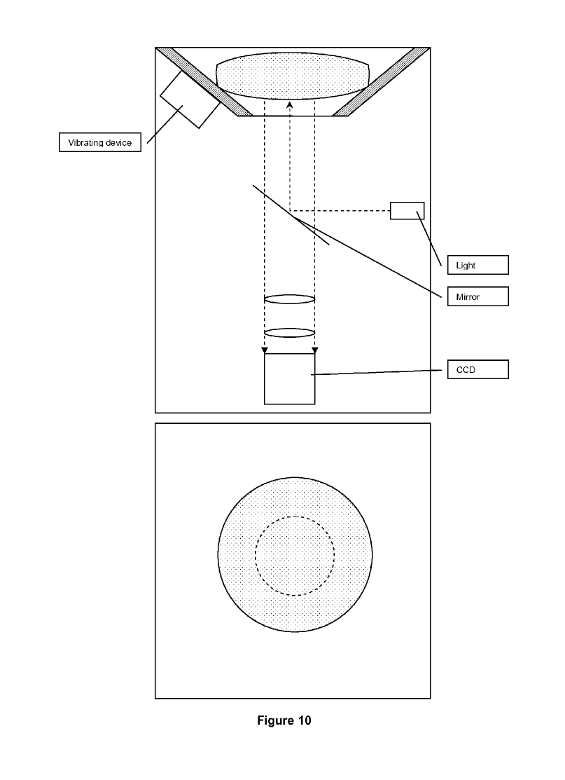

Many imaging system listed above will not lead to reproducible results because the tablet is not flat. Indeed, put on a digital scanner a rounded table may tilt slightly between two different imaging sessions, or even slightly move during the imaging process itself. A custom device can stability the tablet, accounting for its particular shape. For instance a system with a hole smaller then the tablet diameter (possibly vibrating) will lead to a reproducible positioning (as shown in FIG. 10).

Distance Between CCD and Microstructure Surface

Document scanners enable to reliably ensure that the distance between fingerprint surface and CCD will remain constant between several acquisitions. Unfortunately, documents scanner do not provide uniform imaging result across the scanning area (lighting is different between the center and the borders of the scanning window, also when objects are not flat they are some distortions which are different between the center and the borders of the scanning window). A dedicated system can be built such that the distance between CCD and microstructure area is constant between successive snapshots (as shown in FIG. 10).

Location of Imaged Area

It is critical to always image the same area of the tablet. This is a task which is challenging with non-specific devices. One solution consists in having a centering mechanism that ensures that the snapshot will always be taken at the same location on the tablet. For instance, a vibrating system (electro-mechanical) can automatically center the sample. In FIG. 10, a vibrating system is mechanically coupled with the part on which the tablet is put.

Illumination

In order to obtain a reproducible lighting of the sample, any unwanted source of light should be discarded. A closed device with a strong internal illumination system enables to efficiently prevent contamination by uncontrolled and external light sources. In FIG. 10, a lighting system is shown with semi-transparent mirror which enables co-focal illumination.

Depth of Field

If a sample is not totally flat and if the depth of field of the imaging system is small, then it might not possible to obtain the focus on the entire imaged area. One solution consists in using an optical system with a small aperture (larger F-stop number) and increasing consequently exposure time or lighting intensity.

This device could interface with a computer using for instance USB connection, in order to easily control imaging process, lighting and even other positioning functions (like centering for instance).

### Authentication Process

The authentication process consists in comparing an acquired image (test image) of the tablet with a reference image (of the punch or of the tablet). This comparison is performed by digitally computing a value expressing how similar or different are these two digital images (so-called hereafter a similarity measurement). The most straightforward approach consists in computing the mathematical distance between those images, for instance the Mean Square Error. However, in practice in many cases such an approach would fail because it requires a perfect spatial registration of the compared images. Another approach which is more tolerant to errors in the relative positions of both images consists in computing the cross-correlation between the images and measuring, for instance, the signal to noise ratio of the cross-correlation peak (but any other scalar metric of the cross-correlation image can also work, like 1^{st} to 2^{nd} peak ratios, maximum to standard deviation ratios, etc). Three different metrics are explained below and can be used independently or in association for the similarity assessment.

The first metric consists in computing the mean value, the max value and the standard deviation of the cross-correlation image. Then the following formula is used dividing the difference between the max and mean value by the standard deviation

$$S N R = \frac{max - mean}{stdev}$$

The second metric consists in computing the list of the peaks in the image and then dividing the difference between the first peak and the median peak by the difference between the second peak (which is basically noise) and the median peak as in the following formula. A peak in the cross-correlation image is a position which value is higher than all its neighbors.

$$S N R = \frac{{p}^{0} - {p}^{median}}{{p}^{1} - {p}^{median}}$$

The third metric consists in taking the ratio of the max value by the mean value in a normalized picture as in the following formula:

$$S N R = \frac{max - min}{mean - min}$$

This approach will however not work if one of the images is rotated, stretched or more generally suffered from any geometrical transform which is different from a pure translation. More generally those approaches can still work assuming that a way is respectively found for finding the translation and the geometrical alterations between the images, and compensate for those differences before measuring the differences between the images. Finding translation can be accomplished by detecting the contours of the tablets or cross-correlating with a reference image (for instance the logo of the brand engraved in the punch). Finding generalized geometrical transform between images is challenging problem. Typically, an approach consists in identifying several features points and using this information to compute the compensated image. Such feature points can be purposely included on the punch design but it is also possible to use logo or text or any macroscopic identifier on the punch for the same purpose. In particular, if the set of possible transformations is only limited to rotation, the analysis of the Fourier transform of the image is sufficient to compute the rotation angle.

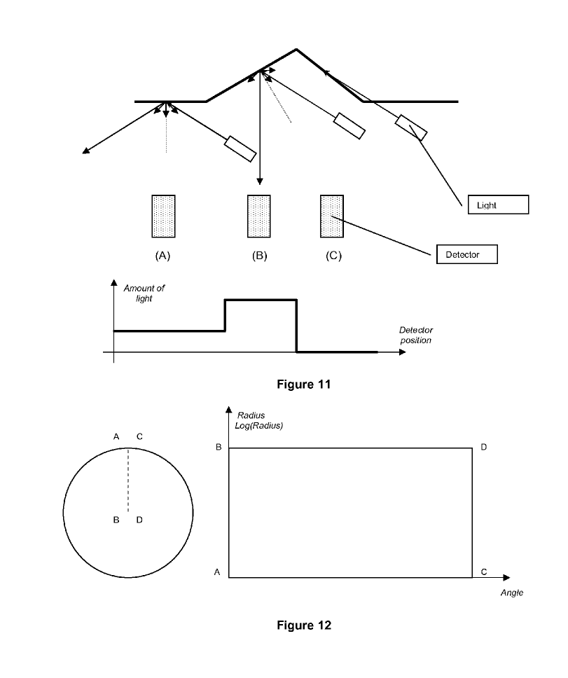

Finally, another approach consists in using a similarity measurement that is not sensitive to geometrical differences (this is the same type of strategy as shown above with the cross-correlation which is not sensitive to translation differences). In the particular case where only rotation differences are considered, one approach consists in unwarping the acquired image as shown in FIG. 12: the images are first cropped in a form of a circle and are then converted into rectangular form, by sweeping the radius of the circle and extracting the part of the image corresponding to the radius into a rectangle, the comparison of the test image and the reference image being carried out by cross-correlating the test rectangle with the reference rectangle. This can be achieved by determining the center (points B/D), the image is cut along AB radius and stretched across a rectangle ABCD. This transform is applied to both the reference image and to the test image of the tablet and the rectangles are then cross-correlated. This cross-correlation enables to successfully perform similarity measurement (using any of the aforementioned scalar metric approaches) of two images even though they have a rotation difference. This approach can also be modified in order to work with images having both differences of rotation angle and difference of scale: for this it is sufficient to cross-correlate the unwarped images ABCD with Log(radius) in the vertical axis. The unwarping can also be done using the Fourier-Mellin transformation, which consists in:
Transforming the acquired picture in the Fourier domain
Keeping only the module of the image
Converting the image in Log-Polar coordinates. Since coordinates cannot be mapped one-to-one pixel, an average has to be computed using for example nearest neighbor, bilinear or bicubic resampling.
Doing the Fourier transform of the resulting picture

This image is invariant to rotation, translation and scale.

Yet another solution for compensating for rotation consists in using a 1D signal a( ) constructed according to the following formula:

α(θ)=∫_{θ}^{R}l(r,θ),dr

Where l( ) is the grayscale intensity of the tablet image (or a flattened version of it) at the location defined in polar coordinates by the distance to the center of the tablet r and an angle ( ) and R is the tablet diameter. Doing this for the reference image creates a reference 1D signal. It then possible for any tablet to compute its 1D signal a′( ) and cross-correlate a( ) and a′( ) to quickly find the rotation angle. Indeed if the tablet comes from the same punch as the reference signal, then the maximum of the cross-correlation signal (as a function of θ) corresponds to the rotation angle difference between the reference and the tested tablet. The tested tablet image can then be rotated by this angle prior to the measure of similarity computation. An absence of cross-correlation peak as a function of θ indicates that the tested tablet does come from a different punch than the reference tablet(s). It should be noted that the same approach can be used by replacing l( ) by the modulus of the Fourier transform of the tablet image.

The various similarity measurements approaches, depending of the types of registration differences, are synthetically summarized in the table below:

| | Requires no | Works with some |
|---|---|---|
| Similarity | translation | translation |
| measurement | difference | differences |
| Requires no | Mean Square Error | Cross-correlation |
| geometrical difference | | |
| Works with some | Cross-correlation | ? |
| geometrical differences | of rotated unwarp | |

Finally, an effective approach consists in a more brute force approach where different geometrical compensations are iteratively tested in order to minimize the differences between images. Although, such approach can potentially lead to compute extremely large sets of transformations, it is possible to greatly reduce the number of combinations to be tested in some cases. First, using cross-correlation will enable to avoid compensating for the translation. Second, for some imaging devices like digital scanners, it can be assumed that there is no scale or stretching differences between the images. In such case, it is only needed to find the rotation angle, and therefore iteratively test for instance 360 degrees and find the best match. The steps of rotation can be computed knowing the robustness to rotation, as described in FIG. 8. For instance, using a step equal to L/2 will guarantee to find a compensation angle. It is of course also possible to work in special trans form domains feature specific characteristics facilitating or enabling detection without geometrical compensation/unwarping, Such domains include log-polar transformation, chirp transforms, etc.

It has to be noted that if the tablet contains a macroscopic identifier, this identifier can be used to retrieve the rotation angle of the test image and therefore rotate the test image so that the rotation angle is compensated.

If the macroscopic identifier is used as an authentication feature, different methods can be used to authenticate the tablet. Different macroscopic identifiers can be taken into account: printing on the tablet, shape of the tablet, engraved shape in the tablet. The shades that will be induced by the lighting system of the acquisition device have to be taken into account when performing the authentication. There is also the possibility to use these shades to create a 3D profile of the tablet. A possibility is to create the 3D profile of the reference using 1 or more tablet to using the shades induced by the lighting system of the acquisition device. In case a macroscopic identifier is used as an authentication feature, the number of reference images is greatly reduced. In fact, the reference image corresponds to the image of a tablet featuring the macroscopic identifier. Only 1 image has to be taken for all the genuine punch die sets featuring the same macroscopic identifier. The comparison between the reference and the test image is performed using Mean Square Error. However any other similarity measurement can be used. The morphology of the differences has to be taken into account. In fact, many small differences can be due to the punch die set and the various processes that are applied to the image, whereas one big difference is likely due to a counterfeiter.

All the above described approach assumes that one single similarity measurement is sufficient for the authentication process. It should be noted that the robustness of this similarity measurement can be greatly enhanced by using several similarity measurements with different level of zooms, with the two sides of the tablets, with pictures acquired from different view angle or with different lighting angles.

As the number of reference images can rapidly increase (between 40 and 60 punch/die set in a single compression machine), especially if the brute force method is used, it is interesting to use a multi-resolution approach to rapidly select a set of references for a possible match. Various methods are described below:

### Decision Tree

A possibility to speed up the detection process is to perform the comparison for images of smaller size to make a first step and then compare only smaller sets of bigger images. For instance if the image size is 1024×1024 and if there are 10,000,000 items in the database of the server, performing all cross-correlations with all references may take a significant amount of time (up to 1 hour in some cases). A detection strategy consists in performing the detection in several stages.

There are different possibilities to obtain a set of smaller images. It is possible to use cropped versions of the references, quantized versions of the references or downsampled versions of the references. Downsampling is preferred instead of cropping. First, downsampling is more resistant in case of dust or other small variations on the image; second, as the positioning is very precise, cropping can lead to the test image and reference image to be completely misaligned. This will not be the case with downsampling. A first stage is performed with downsampled versions of the test and reference images and then the next stage uses larger versions of the tests and references. In a preferred embodiment, the downsampling of the reference image(s) is executed once while the reference image is acquired. The downsampled version of the reference image is stored in the server's database. This approach is illustrated by diagram of FIG. 16: cross-correlations are first computed with a set S0 of X0 references using an image size of 2n×2n pixels (the same method may of course be used for non square images or non integer power of 2 image sizes). A number X12 of cross-correlation images have an SNR over a given threshold t1 and are then selected as candidates for a second test with larger image of size 2n+1×2n+1. The same procedure continues with threshold t2 and with increasing image sizes and thresholds until the original image of size 2n+x+2n+x is reached resulting in one unique candidate X×2=1 which corresponds to the test image. Such strategy is not limited to the case of cross-correlation and can potentially be applied with any matching metric.

A practical example is given in order to illustrate this process. In an experiment n=3 and x=10 were used for cross-correlations of X0=10,000,000 references with a test image. The following number of candidates was then obtained: X12=112539, X22=1234, X32=2, X42=1, X52=1.

Depending on noise characteristics, downsampling down to 8×8 images size can easily be reached.

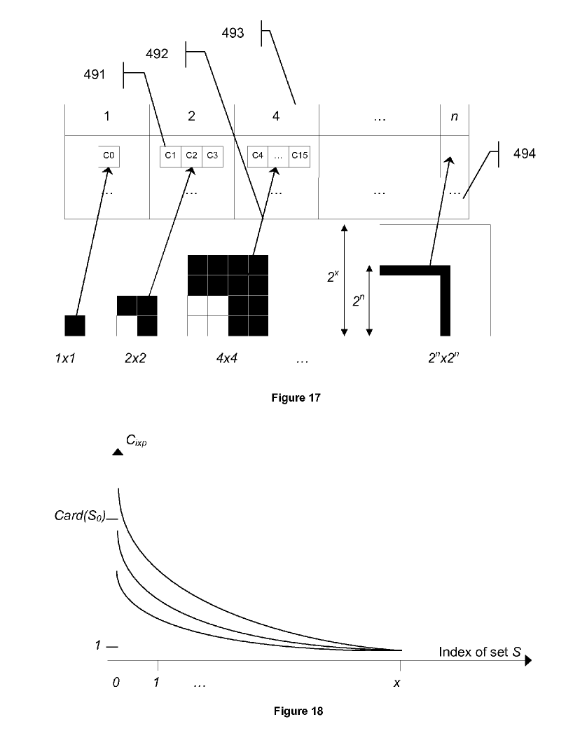

If the correlation is done in the Fourier domain, the coefficients can be stored in a database in an efficient way. It is generally admitted that downsampling an image in the spatial domain will result in a crop in the Fourier domain. Therefore only the coefficients of set Sx are stored in the database. Then for the matching of sets S0 to Sx−1, only some of the coefficients are retrieved from the database. To be accessed efficiently they are split between the different columns. The coefficients for the 2n×2n images can be stored in one column. Then, instead of storing all the coefficients of the 2n+1×2n+1 images, only the remaining ones up to this size can be stored in the next column. The coefficients that are stored in each column 491 of the database table 493 are represented by the black area on FIG. 17. The figure shows that column 1 has only one coefficient (the average value of the image), the column 2 has the 3 following coefficients, the column 3 has 12 coefficients, etc. . . . This approach enables to optimize the required bandwidth for transferring data (492) from the database on the hard disk to the CPU. In fact, all the coefficients of set S0 are transferred but then only the remaining coefficients from the relevant rows are transferred. A new line 494 is allocated in the database for each reference image. Furthermore the multiplied coefficients of the relevant correlations can be stored in order to avoid redundant multiplications. In fact only the coefficients that are displayed in black should be correlated.

### Bayesian Network

A speed up can also be obtained by using a theory based on Bayes probabilities. The notations are the same as those of FIG. 16. Let P(G) be the probability that an item is genuine. For a set Si of cross-correlation, if the SNR is above the given threshold ti+1, then the probability for the image to be already recorded is denoted a. This is modeled by Equation 1.

P(G|SNRᵢ>tᵢ₊₁)=a

i=0, . . . , x−1 Equation 1

It can be stated that if the SNR is some fraction lambda between ti+1 and ti+2, then the probability for the image to be already recorded is b and b>a. This is modeled by Equation 2.

P(G|SNRᵢ>tᵢ₊₁+λ(tᵢ₊₂−tᵢ₊₁))=b

b>a,

i=0, . . . x−1

λε[0,1] Equation 2

All the following assumptions are formulated:
The higher the SNR of a cross-correlation of images of a given size, the higher the SNR of the cross-correlation of images of bigger sizes and the higher the probability of the image to be a recorded one. This is explained by Equation 3.

P(G|SNRᵢ>tᵢ₊₁)=a [symbol] P(G|SNRᵢ₊ⱼ>tᵢ₊ⱼ)=b [symbol] P(G)=c

i=0, . . . , x−1

j>0|i+j≦x

0≦a≦b≦c≦1 Equation 3
For a given set of cross-correlation, if the SNR is under a given threshold, then the probability for the image to be already recorded is 0. This is modeled by Equation 4

P(G|SNRᵢ<tᵢ₊₁)=0

i=0, . . . , x−1 Equation 4
For the cross-correlation from Sx, if the SNR is above the predefined threshold, then the probability for the image to be already recorded is 1.

P(G|SNRₓ>tₓ₊₁)=1 Equation 5

The speed up can be obtained the following way. First all the items of set S0 are correlated together. For each item, if the probability to be genuine is below a, the item is discarded. If it is between a and b, it is put in a set of possible match to be correlated in S1 as for the decision tree algorithm. If the probability to be genuine is more than b, then the picture is directly correlated at higher sizes up to size 2n+x+2n+x. If it is the good match, the algorithm stops. Else it continues to correlate references of set S0, until all have been correlated. Then if the match is still not found the same algorithm is applied for the following sets S1 up to Sx.

### Best Rank

This method is a hybrid one between Decision tree and Bayes networks. The notations are those of FIG. 16. Experimental results show that, for a given set of references, the SNR obtained with low resolution images (typically those of set S0) may significantly differ between imaged items. Furthermore, the rank of the good match is not inevitably the first. Nevertheless, the rank has a smaller variation than the SNR. Experimentally it has been tested to be always in the 5% first. So it can be assumed that if the rank for a given size of one reference is good, there is a higher chance of a match.

So sets can be created by taking into account the references with highest ranks. FIG. 18 is useful to understand this theory. The following notations are used:

x is the number of sets, as shown in FIG. 16,
p is the current set used for cross-correlation.
i is the current iteration
C′ixp is the number of references to take at iteration i from set p, for the next set p+1.

The C′ixp best references are taken at each step. In fact as some of the best references have already been correlated during the preceding iteration, there is no need to correlate them again. Cixp is bigger for smaller size images than for the bigger ones. If after one iteration, the good match is not found, all the Cixp are increased until the good match is found or until a decision is taken that the image is not in the database. As the size of the image has a geometrical growth, the set of remaining references at each set should also follow a geometric law. The idea is to have an increasing common ratio for the geometric progression. Two things are important with this method: the stop criterion as well as the increasing law of the common ratio of the geometrical progression. A geometrical law can be chosen to increase the common ratio of the geometrical progression. The stop criterion is chosen so that the application stops before correlating all the references with a size of 2n+1×2n+1. In fact it is assumed that, if all the references of size 2n+1×2n+1 are correlated, there was no need to use the references of size 2n×2n. More precisely the Cixp are computed as in Equation 6 until i<j. The first line computes the number of references to take at each step. It corresponds to the number of references as computed in the second line minus the references that have already been taken in the preceding iterations. The second line computes the geometrical progression with a common ratio of a. The power corresponds to the iteration number (i) as well as the number of set (x) and the current size (p). The third line simply formulates that at the first iteration no references have already been correlated, therefore the number computed by the second line should be taken into account. The fourth line represents the stop criterion. It tells that the algorithm should stop if S1≧S0.

C′ᵢₓₚ=Cᵢₓₚ−C₍ᵢ₋₁₎ₓₚ

Cᵢₓₚ=a^{i(x-p)}

C′₀ₓₚ=C₀ₓₚ

i=0, . . . j,j|Cⱼₓ₁≦Card(S₀) Equation 6

For example if a=2 and x=5, the following number of references Cixp should be taken at each step. Each row is representing an iteration i. The columns represent index of the set of images. It should be remarked that the last column always contains only one reference, as only one match can be found. In the first row, at i=0, only the best reference is correlated. In the next row, at i=1, 32 references from S0 are taken to correlate in set S1. It can be remarked that the number of reference taken from S0 is growing rapidly. The coverage of the database can be seen in FIG. 18.

TABLE 1

p

| i | 0 | 1 | 2 | 3 | 4 | 5 |
|---|---|---|---|---|---|---|
| 0 | 1 | 1 | 1 | 1 | 1 | 1 |
| 1 | 32 | 16 | 8 | 4 | 2 | 1 |
| 2 | 1'024 | 256 | 64 | 16 | 4 | 1 |
| 3 | 32'768 | 4'096 | 512 | 64 | 8 | 1 |
| 4 | 1'048'576 | 65'536 | 4'096 | 256 | 16 | 1 |

### Neighbors Classifiers

This theory is based on the transitivity of the correlation. It is true that if an image A correlates completely with an image B and if the image B correlates completely with an image C, then A correlates completely with C. But, if an image A doesn't correlate with an image B and if the image B doesn't correlate with an image C, then nothing can be told about the correlation of A and C. The question is then if A correlates to some degree with B and B correlates to some degree with C, what can be told about the correlation of A and C? It can be assumed that the highest the degree of correlation of A and B and of B and C, the highest the probability that A and C also correlate. Therefore, the goal is to compute subset of references that are well correlating together. Then, for the images of group S0 from FIG. 16 instead of correlating the test image with all the references, it is correlated only with the representative of its group. Then a certain number of groups are chosen and the best rank method is used for the other set of images; for S1 up to Sx of FIG. 16.

Another method to reduce the number of references is to select only the reference images corresponding to the same type of tablet than the one to authenticate, for example by using the brand of the tablet.

Before applying the comparison algorithm, it is possible to make the pictures easier to compare by applying a so called flattening process. The goal of this process is to highlight the structure of the tablets to accurately compare them. There are many possibilities to perform this flattening process:
Gaussian flattening: taking the difference between the picture and its Gaussian frequently lowpassed version
Other flattening: Other than Gaussian low pass filtering of the image
Local histogram equalization

1. A method to authenticate a genuine tablet manufactured by compressing powder between a punch and die set comprising the steps of:
acquiring at least one reference image of the face of the punch and die set having the microstructure formed thereon, or of a face of a first tablet formed by the punch and die set and corresponding to the microstructure;
acquiring at least one test image of a second tablet to be authenticated;
computing a level of similarity by an electronic device between the at least one test image and the at least one reference image; and
comparing the computed level with a threshold value so as to determine if the second tablet is genuine.

2. The method of claim 1, wherein the at least one reference image is an image of the first tablet, and wherein a coating is applied on the first tablet, and the acquisition of the at least one reference image is performed before the coating is applied.

3. The method of claim 1, wherein the at least one reference image is an image of the first tablet, and wherein a coating is applied on the first tablet, and the acquisition of the at least one reference image is performed after the coating is applied.

4. The method of claim 1, wherein the at least one reference image is an image of the first tablet, wherein the first tablet is formed by compressing powder using a plurality of punch and die sets, each of the plurality of punch and dies sets having a face with a microstructure formed thereon, and wherein the acquisition of the at least one reference image comprises the steps of acquiring at least one reference image of each face of the tablet corresponding to a face of one of the punch and die sets containing a microstructure.

5. The method of claim 1, further comprising the step of creating a microstructure on the punch and die set, wherein the step of creating a microstructure on the punch and die set comprises the step of:
acquiring a preliminary image of a face of the punch and die set on which the microstructure is to be formed or of a face of a tablet formed using the punch and die set, the face of the tablet corresponding to the face of the punch and die set on which the microstructure is to be formed;
computing a frequency spectrum of the preliminary image;
identifying differences between the computed frequency spectrum and a predefined frequency spectrum; and
modifying the punch and/or the die microstructure in order to compensate for the differences.

6. The method of claim 1, wherein the step of computing the level of similarity comprises:
successively applying a rotation angle between the at least one test image and the at least one reference image, and producing a level of similarity for each rotation angle by comparing the two images; and
keeping the maximum value as resulting level of similarity.

7. The method of claim 1, further comprising the steps of:
generating a plurality of downsampled reference, images of the at least one reference image, by downsampling the previously downsampled reference image;
generating a plurality of downsampled test images by downsampling the previously downsampled test image;
by starting from the lowest image size, comparing the downsampled version of the at least one test image with the corresponding downsampled at least one reference image stored in the reference database, and selecting downsampled reference images for which a level of similarity of said comparison is above a predefined threshold, and repeating the comparison with reference images of a larger size having been selected; and
comparing the test image with the reference images for only the selected downsampled reference images.

8. The method of claim 1, wherein the microstructure of the punch and die set is created so that the grain of the powder can follow the profile of the microstructure.

9. The method of claim 1, wherein the at least one test image and the at least one reference image are cropped to form a circle, the cropped images are then converted into rectangular form, by sweeping the radius of the circle and extracting the part of the image corresponding to the radius into a rectangle, the comparison of the at least one test image and the at least one reference image being carried out by cross-correlating the test rectangle with the reference rectangle.

10. The method of claim 1, wherein the step of computing the level of similarity comprises:
transforming the at least one reference image and the at least one test image into the frequency domain so as to obtain a frequency domain reference image and a frequency domain test image;
computing a module of each frequency domain image;
computing the log polar transform of the module of each frequency domain image in order to obtain a test log polar transformed and a reference log polar transformed; and
comparing the reference log polar transformed with the test log polar transformed to determine the level of similarity.

11. The method of claim 1, comprising a step of introducing an identifier of the tablet to be authenticated, and selecting the at least one reference image related only to the identifier of the tablet for the step of computing the level of similarity.

12. The method of claim 1, when the authentication is enhanced by a macroscopic feature.

13. Pills or tablets manufactured according to the method of claim 18.

14. Pills or tablets according to claim 13, further comprising a visible macrostructure.

15. Pills or tablets according to claim 13, further comprising a coating.

16. Pills or tablets according to claim 14, further comprising a coating.

17. A method for creating a microstructure on a punch and die set, the method comprising:
selecting a face of the punch and die set on which the microstructure is to be formed;
acquiring a preliminary image of a face of the punch and die set on which the microstructure is to be formed or of a face of a tablet formed using the punch and die set, the face of the tablet corresponding to the face of the punch and die set on which the microstructure is to be formed;
computing a frequency spectrum of the preliminary image;
identifying differences between the computed frequency spectrum and a predefined frequency spectrum; and
modifying the microstructure of the face of the punch and die set in order to compensate for the differences.

18. A method for manufacturing a pill or tablet, the method comprising:
compressing powder using the punch and die set of claim 17 to form a pill or tablet with a face corresponding to the microstructure.
